(19) United States
(12) Patent Application Publication
(10) Pub. No.: US 2018/0242849 A1
(43) Pub. Date: Aug. 30, 2018

(54) ULTRASOUND RECEIVING APPARATUS

(71) Applicant: CANON KABUSHIKI KAISHA, Tokyo (JP)

(72) Inventors: Kazuhiko Fukutani, Yokohama-shi (JP); Takuro Miyasato, Tokyo (JP); Fumitaro Masaki, Brookline, MA (US); Nobuhito Suehira, Tokyo (JP)

(73) Assignee: Canon Kabushiki Kaisha, Tokyo (JP)

(21) Appl. No.: 15/756,226

(22) PCT Filed: Aug. 12, 2016
(86) PCT No.: PCT/JP2016/003723

§ 371 Date: Feb. 28, 2018

(30) Foreign Application Priority Data
Sep. 4, 2015 (JP) 2015-175021
Jul. 28, 2016 (JP) 2016-148213

(51) Int. Cl. A61B 5/00; G01N 29/24; G01N 29/28; G01N 29/22

(52) U.S. Cl. CPC A61B 5/0095; G01N 29/2418; G01N 2291/106; G01N 29/223; G01N 29/28

(57) ABSTRACT

An ultrasound receiving apparatus of the present invention includes an ultrasound generation member. The ultrasound generation member is a sheet-shaped light absorbing member which is in acoustic contact with an acoustic liquid disposed between the subject and the ultrasound receiving array so as to acoustically combine the subject and the ultrasound receiving array, and is stretched in the acoustic liquid to be located between the subject and the ultrasound receiving array.

### TECHNICAL FIELD

The present invention relates to an ultrasound receiving apparatus which acquires information about a subject using ultrasound.

### BACKGROUND ART

In the medical field, ultrasound imaging apparatuses which image information in a subject by irradiating the subject with a photoacoustic wave (an ultrasound) induced upon irradiation of an ultrasound generation member disposed outside the subject with light, such as laser, and receiving ultrasound scattered waves scattered from the subject are under study. This technology is called photoacoustic induced ultrasound imaging (PA-Induced US) for the distinction from ordinary pulse echo ultrasound imaging in which acoustic waves electrically generated by an acoustic wave transmission element are used as transmission waves. In the photoacoustic induced ultrasound imaging, unlike ordinary pulse echo ultrasound imaging, the ultrasound generation member disposed outside the subject is irradiated with pulsed light emitted from a light source and the subject is irradiated with the ultrasound induced by the irradiation of the ultrasound generation member with pulsed light. Receiving the ultrasound scattered from the subject and imaging subject information on the basis of reception signals are common to the photoacoustic induced ultrasound imaging and the pulse echo ultrasound imaging. Scattering of the ultrasound here includes reflection of the ultrasound.

In the photoacoustic induced ultrasound imaging, spherical optical absorbers are usually used in order to avoid the influence of, for example, a phase delay produced when the ultrasound scattered from the subject pass through the ultrasound generation member. NPL 1 discloses acquiring a high resolution ultrasound image by using carbon microspheres as spherical optical absorbers and, thereby, generating broadband spherical waves.

### CITATION LIST

### Non Patent Literature

NPL 1: Thomas Felix Fehm, Xose Luis Dean-Ben, Daniel Razansky, “Hybrid optoacoustic and ultrasound imaging in three dimensions and real time by optical excitation of a passive element,” 2015, Proceeding of SPIE Vol. 9323 93232X

### SUMMARY OF INVENTION

NPL 1 does not have detailed description of arrangement of the spherical optical absorbers. The present inventors have found a problem that, depending on the arrangement of the spherical optical absorbers, a shear wave component having a propagation speed which is different from that of a longitudinal wave component upon entrance of an ultrasound in the spherical optical absorbers or their tension member may be generated and propagate, whereby accuracy of the acquired image may be decreased. The same phenomenon occurs in an ultrasound receiving apparatus which does not image information; for example, an ultrasound receiving apparatus which calculates information about a subject from a received result and displays a calculation result.

The present invention provides a photoacoustic induced ultrasound receiving apparatus which hardly causes a shear wave component upon entrance of an ultrasound in an ultrasound generation member.

### Solution to Problem

The present invention provides an ultrasound receiving apparatus including: an ultrasound generation member configured to generate an ultrasound upon irradiation of light from a light source; an ultrasound receiving array configured to receive the ultrasound propagating from a subject and output a reception signal; and a signal processor configured to acquire acoustic characteristic value information about the subject from the reception signal, wherein the ultrasound generation member is a sheet-shaped light absorbing member which is in acoustic contact with an acoustic liquid disposed between the subject and the ultrasound receiving array so as to acoustically combine the subject and the ultrasound receiving array, and is stretched in the acoustic liquid to be located between the subject and the ultrasound receiving array.

Other aspects of the present invention will become apparent from the embodiments described below.

### Advantageous Effects of Invention

According to the present invention, a photoacoustic induced ultrasound receiving apparatus which hardly causes a shear wave component upon entrance of an ultrasound in an ultrasound generation member can be provided.

Further features of the present invention will become apparent from the following description of exemplary embodiments with reference to the attached drawings.

### BRIEF DESCRIPTION OF DRAWINGS

FIG. 1 is a diagram schematically illustrating an exemplary configuration of an ultrasound receiving apparatus according to an embodiment of the present invention.

FIG. 2A is a diagram schematically illustrating an exemplary ultrasound generation member of an ultrasound receiving apparatus according to an embodiment of the present invention.

FIG. 2B is a diagram schematically illustrating an exemplary ultrasound generation member of an ultrasound receiving apparatus according to an embodiment of the present invention.

FIG. 2C is a diagram schematically illustrating an exemplary ultrasound generation member of an ultrasound receiving apparatus according to an embodiment of the present invention.

FIG. 2D is a diagram schematically illustrating an exemplary ultrasound generation member of an ultrasound receiving apparatus according to an embodiment of the present invention.

FIG. 3A is a diagram schematically illustrating propagation of ultrasound according to an embodiment of the present invention.

FIG. 3B is a diagram schematically illustrating propagation of ultrasound according to an embodiment of the present invention.

FIG. 3C is a diagram schematically illustrating propagation of ultrasound according to an embodiment of the present invention.

FIG. 4A is a diagram illustrating exemplary distribution information data of acoustic characteristic values according to an embodiment of the present invention.

FIG. 4B is a diagram illustrating exemplary distribution information data of acoustic characteristic values according to an embodiment of the present invention.

### DESCRIPTION OF EMBODIMENTS

Hereafter, embodiments of the present invention are described in detail with reference to the drawings. The same components are denoted by the same reference numerals and are not described repeatedly. In the ultrasound receiving apparatus of the present embodiment, an ultrasound generation member which generates an ultrasound upon light irradiation functions as a plane sound source upon light irradiation, and generates an ultrasound which is substantial plane wave. Therefore, since energy attenuation of the ultrasound is less easily caused even at positions far from the ultrasound generation member in a path in which geometric attenuation from the sound source is suppressed and echo ultrasound reflected from the subject propagate, an S/N ratio of an acoustic wave signal is less easily lowered. The substantial plane wave here includes a perfect plane wave.

### Basic Configuration

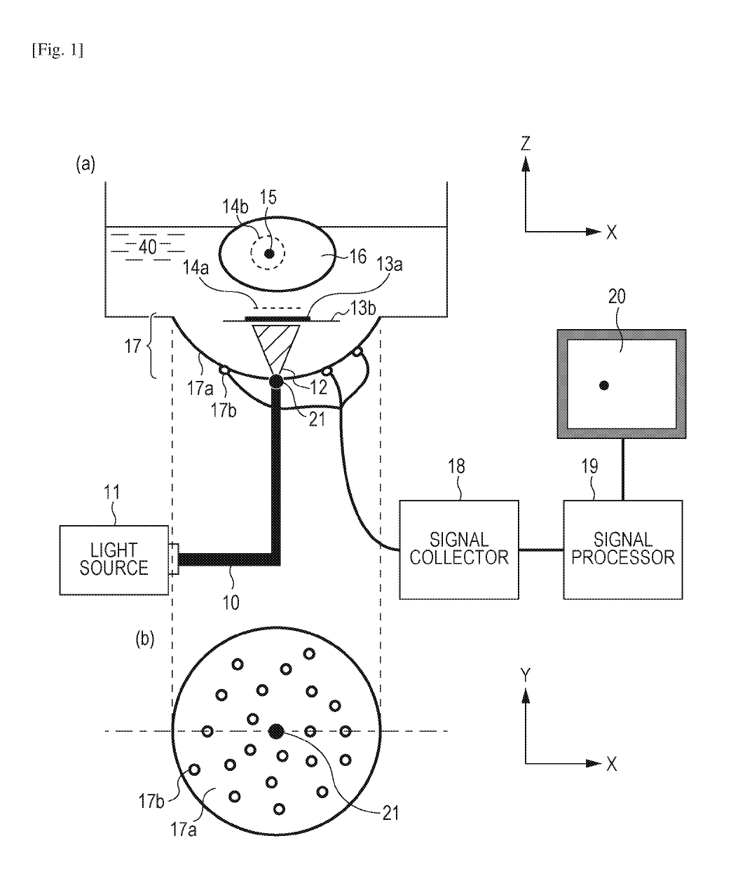

A configuration of an ultrasound receiving apparatus according to the present embodiment is described with reference to FIG. 1. The ultrasound receiving apparatus of the present embodiment is an ultrasound imaging apparatus which receives ultrasound reflected or scattered from a subject (hereafter, referred to as “backscattered ultrasound”), acquires acoustic characteristic value distribution information inside the subject on the basis of a received result, and images the information. The acoustic characteristic value distribution information generally represents acoustic impedance difference distribution, scattering intensity distribution, sound velocity distribution, sound attenuation distribution, or distribution data having values related to these distributions. The acoustic characteristic value information data here may be referred to as image data.

The ultrasound imaging apparatus of the present embodiment includes, as a basic hardware configuration, a light source 11, an ultrasound generation member 13a, a tension member 13b which keeps the ultrasound generation member 13a stretched at a position spaced from a subject 16, an ultrasound receiving array 17, and a signal processor 19. Pulsed light 12 emitted from the light source 11 is irradiated from an irradiation portion 21 while being processed into a desired light distribution shape by an optical system 10, and is made to irradiate the ultrasound generation member 13a provided outside the subject 16. The ultrasound generation member 13a is a sheet-shaped sound source disposed spaced from the subject 16, is located between the subject 16 and the ultrasound receiving array 17, and is stretched by the tension member 13b in an acoustic liquid 40. With this configuration, shearing stress is reduced and generation of a shear wave is reduced also in the acoustic liquid 40 and the ultrasound generation member 13a located on a propagation path. Therefore, even if the ultrasound generation member 13a is disposed on the propagation path, an echo wave in which longitudinal wave is dominant can be received and thus artifact caused by the shear wave is reduced. Further, since the ultrasound generation member 13a is stretched in a sheet shape in the acoustic liquid 40, geometric attenuation is reduced and information about a deep part of the subject 16 can be acquired even if the subject 16 is elongated in the direction of the propagation path. When ultrasound 14a which propagate through the subject 16 are made to irradiate an ultrasound scatterer 15 located inside the subject 16, the ultrasound 14a is scattered (and reflected) from the ultrasound scatterer 15, thereby generating backscattered ultrasound 14b. After the backscattered ultrasound 14b is received by the ultrasound receiving array 17, the backscattered ultrasound 14b is amplified and converted into digital signals by a signal collector 18. The signals are subject to a predetermined process in a signal processor 19, and are finally converted into image data of the subject 16 (i.e., acoustic characteristic value distribution information data). In the present embodiment, the image data of the subject 16 is imaged and finally displayed on a display device 20.

### Light Source 11

The light source 11 generates the pulsed light 12, with which the ultrasound generation member 13a is irradiated. The light source 11 may be provided integrally with the ultrasound imaging apparatus as in the present embodiment, or may be provided separately from the ultrasound imaging apparatus. The light source 11 is desirably a pulsed light source capable of emitting pulsed light 12 of the order of from several nanoseconds to several hundreds of nanoseconds as irradiation light. Specifically, the pulse width about 1 to 50 nanoseconds is used to efficiently generate an optical ultrasound. Although laser is desirably used as the light source 11 for its large output, other light sources, such as light emitting diode, may also be used. If laser is used as the light source 11, solid-state laser, gas laser, fiber laser, dye laser semiconductor laser, and other lasers may be used. Timing of irradiation, waveforms, intensity and the like are controlled by an unillustrated light source control unit. The wavelength of the pulsed light 12 with which the used light source 11 irradiates the ultrasound generation member 13a is desirably absorbed strongly by the ultrasound generation member 13a.

### Optical System 10

The pulsed light 12 generated by the light source 11 is processed into a desired light distribution shape by the optical system 10 and is guided to the ultrasound generation member 13a.

An example in which optical fiber is used as an optical waveguide is illustrated in FIG. 1, but the configuration of the optical system is not limited thereto. The optical system 10 may be constituted by optical components, such as optical fiber which guides light, a mirror which reflects light, a lens which condenses, extends, or changes shapes of light, and a diffuser which diffuses light. These optical components are used alone or in combination. Any optical components may be used as long as the pulsed light 12 emitted from the irradiation portion 21 is made to irradiate the ultrasound generation member 13a in a desired shape. In the present embodiment, in order that the pulsed light 12 uses the ultrasound generation member 13a as a plane sound source, the pulsed light 12 is extended so that an area irradiated with light (hereafter, “irradiated area”) of the ultrasound generation member 13a has a certain amount of area by condensing light with a lens or diffusing light with a diffuser. The area of the irradiated area (hereafter, “irradiation area”) affects the characteristic of a substantial plane wave generated from the plane sound source. For example, if the irradiation area is small with respect to the frequency band of the generated ultrasound, the generated ultrasound diffuses. With this configuration, energy of the ultrasound with which the subject 16 is irradiated decreases as the distance from the ultrasound generation member 13a becomes longer. Therefore, energy of the ultrasound which reaches the deep part of the subject 16 decreases and an S/N ratio of information about the deep part of the subject and an S/N ratio of an image generated on the basis of the information are lowered. Therefore, the irradiation area is desirably adjusted to such an extent that the ultrasound having sufficient energy propagates through a region of interest (i.e., an area from which information is to be acquired). If the distance between the light source 11 and the ultrasound generation member 13a is short and it is possible to irradiate the ultrasound generation member 13a with the light emitted from the irradiation portion 21 directly, the optical system 10 is unnecessary.

### Ultrasound Generation Member 13a

The pulsed light 12 emitted from the irradiation portion 21 is guided to the ultrasound generation member 13a stretched in the acoustic liquid 40 via the optical system 10. The ultrasound generation member 13a absorbs the irradiated light and generates a photoacoustic wave which is an ultrasound. The sound pressure P₀ of the generated ultrasound is expressed by the following equation.

[Math.1]

P₀=Γ·μₐ·Φ (1)

In Expression (1), Γ denotes Gruneisen coefficient of the ultrasound generation member 13a and is a coefficient with which the absorbed energy is converted into pressure, μₐ denotes an optical absorption coefficient of the ultrasound generation member 13a, and φ denotes an amount of light with which the ultrasound generation member 13a is irradiated. The optical absorption coefficient μₐ here is the optical absorption coefficient on the center wavelength of the pulsed light 12 emitted from the irradiation portion 21. As is known from Expression (1), using a material with a higher Gruneisen coefficient is desirable to increase sound pressure generated by the ultrasound generation member 13a. Since the Gruneisen coefficient is proportional to the thermal expansion coefficient (the linear expansion coefficient or the volume expansion coefficient), it is desirable to use a material with a higher linear expansion coefficient 3 basically. For example, it is more desirable to use rubber such as silicone (the linear expansion coefficient: 200×10⁻⁶/K or less) than plastic such as high-density polyethylene (the linear expansion coefficient: 100×10⁻⁶/K or less) and solids, such as carbon, glass, and metal (the linear expansion coefficient: 10×10⁻⁶/K or less). Especially, as described below, the linear expansion coefficient 3 is desirably greater than 10×10⁻⁶/K.

β>10×10⁻⁶/K (2)

The optical absorption coefficient μₐ can be adjusted easily by mixing ink or the like with the material. Therefore, any material may be used basically as long as the material has sufficient absorption with respect to the wavelength of incident light or the optical absorption coefficient of the material can be adjusted.

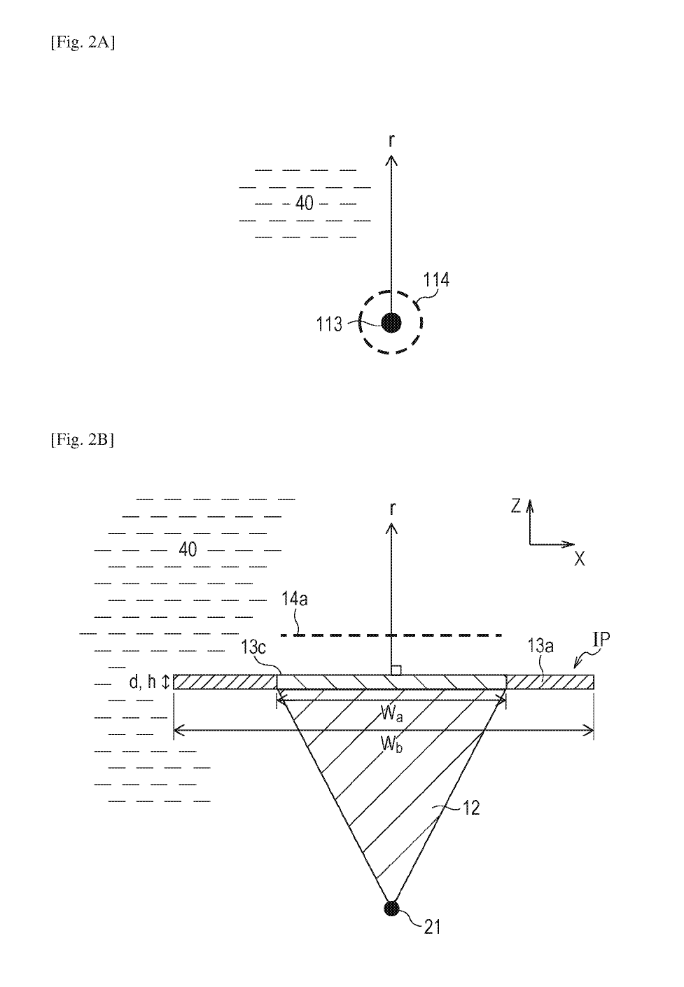

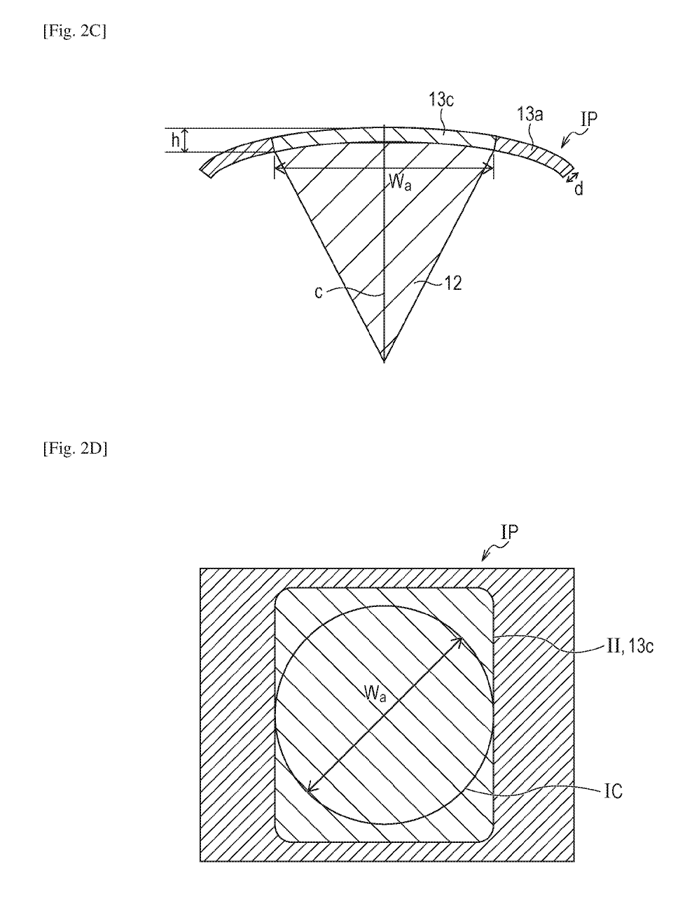

Spherical absorbers which generate spherical waves are used an ultrasound generation member in NPL 1, whereas a member which becomes a plane sound source upon light irradiation is used as the ultrasound generation member in the present embodiment. In the present embodiment, the S/N ratio of the information about the deep part of the subject can be increased. The reason is as follows. As illustrated in FIG. 2A, a spherical wave 114 is generated upon light irradiation from the spherical ultrasound generation member 113, i.e., the point sound source. The energy of the spherical wave generated from the point sound source is diffusion-attenuated at 4πr² as the propagation distance r in the propagating direction becomes longer. Similarly, the energy diffusion-attenuates at 4πr from an unillustrated linear sound source. Such attenuation of the acoustic wave depending on the propagation distance r is generally referred to as geometric attenuation. That is, intensity of the propagating ultrasound is lowered as the distance from the spherical ultrasound generation member 113 becomes longer. If a member which becomes a plane sound source upon irradiation of pulsed light 12, e.g., a sheet member (a membrane member) is used as the ultrasound generation member 13a as illustrated in FIG. 2B, the generated ultrasound 14a is a wave propagating substantially planarly (a substantial plane wave). Therefore, energy of the ultrasound hardly diffusion-attenuates as the distance from the ultrasound generation member 13a becomes longer. That is, intensity of the propagating ultrasound hardly changes as the distance from the ultrasound generation member 13a becomes longer. Therefore, ultrasound characteristic information with high contrast can be imaged even at the deep part of the subject 16.

The plane sound source here is a sound source which generates a plane wave and has an effect to make the generated plane wave propagate through an acoustic medium without causing any substantially geometric attenuation. Specifically, if the width Wₐ of the irradiated area 13c is 10 or more times of the height d as illustrated in FIG. 2B, the ultrasound generation member 13a is considered to function as the plane sound source. This means that, as illustrated in FIG. 2D, a diameter Wₐ of an imaginary circle IC inscribed in a virtual irradiation surface II which is the irradiated area 13c irradiated with the pulsed light 12 in the ultrasound generation member 13a orthogonally projected onto a virtual plane IP of which irradiation optical axis c is the normal line is 10 or more times of the thickness d in the direction parallel to the irradiation optical axis. In the present embodiment, the thickness d of the ultrasound generation member 13a and the height h in the propagating direction are the same. FIG. 2C illustrates a state in which the ultrasound generation member 13a extends with a curvature. In the present embodiment, the thickness d of the ultrasound generation member 13a and the height h in the propagating direction are not necessarily the same. However, if a difference between the height h and the thickness d (h−d) in the propagating direction in the irradiated area 13c is sufficiently small with respect to the distance r from the unillustrated subject 16, the ultrasound generation member 13a corresponding to the irradiated area 13c can be considered to be a plane sound source. The curvature of the ultrasound generation member 13a that can be considered to be the plane sound source should be (h−d)/r is equal to or smaller than 0.01. Although it depends on the frequency of the ultrasound to be generated, if an ultrasound of from several MHz to about 10 MHz is used, the width Wₐ of the irradiated area 13c is desirably 20 or more times, and more desirably 30 or more times, of the height h. Since the length of the irradiated area 13c in the width direction of the irradiated area 13c (i.e., the width Wₐ of the irradiated area 13c) is 10 or more times of the height h of the irradiated area 13c, the length of the irradiated area 13c in the longitudinal direction of the irradiated area 13c is also 10 or more times of the height h of the irradiated area 13c. For example, if the irradiated area 13c is round in shape, the length of the irradiated area 13c in the width direction and the length of the irradiated area 13c in the longitudinal direction are the same, and both are 10 or more times of the height h of the irradiated area 13c. The shape of the ultrasound generation member 13a of which generated ultrasound becomes a plane sound source as described above is desirably a shape of a member of which generated ultrasound becomes a plane sound source, such as a sheet-shape (a film-shape). If the ultrasound generation member 13a has a sheet shape, for example, it is desirable that the width w_{b} of the ultrasound generation member 13a is sufficiently large to the width wₐ (which is equal to the width of the incident light) of the irradiated area 13c which is irradiated with light as illustrated in FIGS. 2B and 2C. Since the incident light is converted into an ultrasound efficiently, the plane wave is generated efficiently. If the optical absorption coefficient of the ultrasound generation member 13a is denoted by μₐ, the thickness d of the ultrasound generation member 13a desirably satisfies the following Expression.

$$[ Math .2 ]$$

$$d \geq \frac{1}{{μ}_{a}} \qquad ( 3 )$$

Expression (3) shows that the thickness d of the ultrasound generation member 13a is desirably greater than an inverse of the optical absorption coefficient μₐ. Therefore, the ultrasound generation member 13a can absorb almost all the energy of the incident light, and propagation of light in the direction of the subject 16 and generation of the photoacoustic wave on the surface and inside of the subject 16 can be reduced. When a part of the light with which the ultrasound generation member 13a is irradiated propagates from the light source 11 to the subject 16, the photoacoustic wave is generated from the subject 16, which occurs artifact that may cause image deterioration.

The frequency domain of the ultrasound generated by the ultrasound generation member 13a is determined by the optical absorption coefficient μₐ if the thickness d satisfies Expression (3). The maximum main frequency fₘₐᵢₙ of the generated ultrasound is expressed by the following relational expression if the sound velocity is denoted by c₁.

[Math.3]

fₘₐᵢₙ≈c₁·μₐ (4)

Expression (4) shows that it is necessary to make the optical absorption coefficient of the ultrasound generation member 13a high in order to increase the maximum main frequency of the ultrasound to be generated. If the maximum frequency receivable by an ultrasound receiving element is denoted by fₘₐₓ, fₘₐᵢₙ is desirably larger than fₘₐₓ because the generated ultrasound is received efficiently.

Although an example in which the ultrasound generation member 13a has a flat sheet shape is illustrated in FIG. 2B, a sheet having a degree of curvature as illustrated in FIG. 2C may be used as the ultrasound generation member 13a as long as it functions as a plane sound source. If the ultrasound generation member 13a having a curvature is used, the ultrasound generation member 13a generates an ultrasound which is collected (in a case in which the generation member is convex to a light irradiation surface) or diffused (in a case in which the generation member is concave to the light irradiation surface) depending on the curvature. Even in the case in which the ultrasound is to be diffused, if the curvature of the ultrasound generation member 13a is so small that the generation member may function as a plane sound source, the influence of the diffusion is sufficiently negligible as compared with the case of the spherical wave. For example, if the ultrasound generation member 13a is concave to the light irradiation surface, the ultrasound can be made to propagate in an area greater than the irradiated area. Then an area greater than the irradiated area can be imaged. If the ultrasound generation member 13a is convex to the light irradiation surface, an imagable area is smaller than the irradiated area, but an SN ratio of the image at the deep part of the subject is further increased since the propagation energy is increased.

The ultrasound generation member 13a is desirably made from a material having a small Young's modulus (or modulus of rigidity), such as rubber (Young's modulus: about 0.0015 GPa). In isotropic materials (i.e., materials without anisotropy), modulus of rigidity G and Young's modulus E are expressed by Expression (5).

$$[ Math .4 ]$$

$$G = \frac{E}{2 ( 1 + v )} \qquad ( 5 )$$

In Expression (5), v denotes the Poisson's ratio. Expression (5) shows that the Young's modulus is proportional to the modulus of rigidity. The modulus of rigidity is a kind of elastic modulo, and is a physical property value that determines difficulty in deformation due to shear force. The modulus of rigidity is also called shear modulus, shear modulus, transverse elasticity modulus, and the second Lame constants.

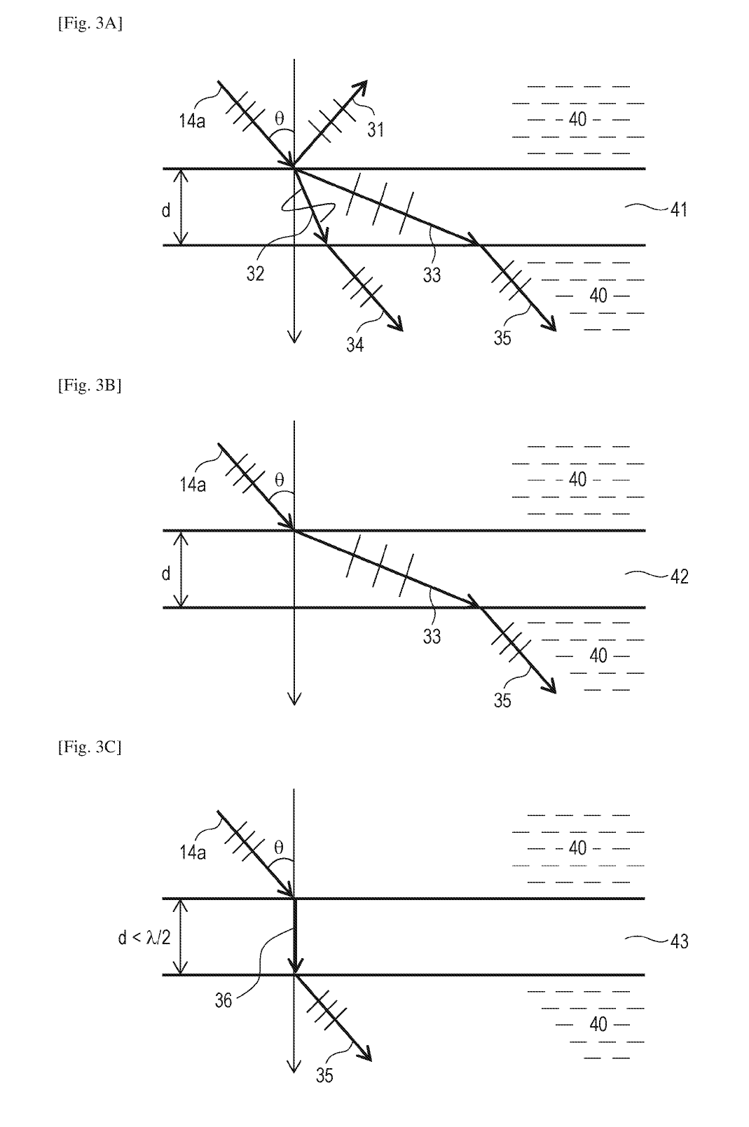

Hereafter, a reason for which materials having small Young's modulus (or modulus of rigidity) are desirably used is described. For example, as illustrated in FIG. 3A, propagation of the ultrasound when the backscattered ultrasound 14b enters high-density polyethylene 41 (Young's modulus: about 1.4 GPa) which is the plastic having the Young's modulus larger than that of rubber through the acoustic liquid 40 will be considered. High-density polyethylene is plastic having the Young's modulus higher than that of rubber. When the backscattered ultrasound (the longitudinal wave) 14b enters at or above a certain incidence angle (θ), shear force is produced within the high-density polyethylene 41 and a shear wave 32 is generated within the high-density polyethylene 41 (longitudinal wave-shear wave conversion). Especially at or above the total reflection angle of the longitudinal wave, propagating in the shear wave becomes dominant in the propagation of the ultrasound within the high-density polyethylene 41. The shear wave 32 is again converted into the longitudinal wave 34 on an interface with the acoustic liquid 40 on the opposite side, propagates through the acoustic liquid 40, and is received by receiving elements 17b. A longitudinal wave 33 refracted on the interface between the acoustic liquid 40 and the high-density polyethylene 41 is again refracted on the interface between the acoustic liquid 40 and the high-density polyethylene 41 on the opposite side, propagates through the acoustic liquid 40 as a longitudinal wave 35, and is received by the receiving elements 17b. The sound velocities of the longitudinal wave 33 and the shear wave 32 of the ultrasound propagating through a solid object including plastic usually differ significantly. The shear wave 32 has lower sound velocity. Therefore, the backscattered ultrasound 14b passing through the ultrasound generation member 13a has greatly different propagation time (or phase) depending on the propagation process in which the shear wave or the longitudinal wave are dominant. The same phenomenon occurs when a large number of spherical structures are arranged on a planar member formed from a material having a large Young's modulus, such as high-density polyethylene, in order to generate a substantial plane wave using the spherical structures. Therefore, in order to image the acoustic characteristic information inside the subject 16 with high accuracy, it is necessary to employ an image reconstruction technique in consideration of such a complicated physical phenomenon. That is, a delay calculation using average sound velocity as described in NPL 1 may provide an image with low accuracy.

In a case in which rubber 42 having small Young's modulus is used as illustrated in FIG. 3B, since shear stress is small inside the rubber 42, generation of a shear wave by shear force (longitudinal wave-shear wave conversion) is reduced significantly, and the longitudinal wave-shear wave conversion and shear wave propagation inside the rubber 42 is substantially negligible. That is, the ultrasound received by the receiving elements 17b can be considered to be the ultrasound which has propagated through the acoustic liquid 40 and the rubber 42 as the longitudinal wave (14b, 33 and 35). Since the material having small Young's modulus has acoustic characteristics that are similar to those of the acoustic liquid 40, such as water, the angle at which the total reflection of the longitudinal wave occurs (the total reflection angle) can be made larger than that of plastic or other materials. Therefore, the backscattered ultrasound 14b entering the ultrasound generation member 13a at various angles is received effectively with a receiver. Thus, by using, for example, rubber having the small Young's modulus as the ultrasound generation member 13a, it is possible to acquire a high definition ultrasound image even if a general image reconstruction algorithm as described in NPL 1 is used. Therefore, the material having the Young's modulus E of 0.1 GPa or less is desirably used as the ultrasound generation member 13a of the present embodiment as expressed by Expression (6).

[Math.5]

E≤0.1 GPa (6)

The phase change due to longitudinal wave-shear wave conversion and shear wave propagation within the ultrasound generation member 13a can be decreased by reducing the thickness d of ultrasound generation member 13a even in a case in which an ultrasound generation member having the Young's modulus of 0.1 GPa or greater, such as plastic, is used. Specifically, if the maximum frequency of the ultrasound receivable by the receiving elements is denoted by fₘₐₓ and the sound velocity of the longitudinal wave in the ultrasound generation member 13a is denoted by c₁, the minimum receiving wavelength λ of the ultrasound of the frequency fₘₐₓ within the ultrasound generation member 13a is expressed by the following Expression (7). The maximum frequency fₘₐₓ of the ultrasound receivable by the receiving elements here is the maximum frequency having half the sensitivity of the frequency having the maximum sensitivity. The minimum receiving wavelength here refers to as a wavelength corresponding to the maximum frequency fₘₐₓ.

λ=c1/fₘₐₓ (7)

If the thickness d of the ultrasound generation member 13a is sufficiently small to the ultrasound of the minimum receiving wavelength λ propagating through the ultrasound generation member 13a, the influence of the shear wave propagation with respect to the ultrasound of the frequency fₘₐₓ which has passed through the ultrasound generation member 13a is reduced. Typically, as expressed by the following Expression (8), if the thickness d is smaller than one half of the minimum receiving wavelength λ of the valid receiving band of an ultrasound receiving array 17, the influence of the shear wave propagation is negligibly small.

$$[ Math .6 ]$$

$$d < \frac{λ}{2} \qquad ( 8 )$$

Expression (8) shows that, if the thickness d of the ultrasound generation member 13a is smaller than one half of the minimum receiving wavelength λ of the valid receiving band of the ultrasound receiving array 17, the ultrasound (the longitudinal wave) propagating from the acoustic liquid 40, such as water, to the ultrasound generation member propagates through an ultrasound generation member 43 as an evanescent wave 36 as illustrated in FIG. 3C. The ultrasound 14a is not converted into a shear wave, but passes through the ultrasound generation member 43, and the longitudinal wave 35 is received by the receiving elements 17b. Therefore, the total reflection angle of the ultrasound (the longitudinal wave) can be made larger than that in the case in which the thickness of the ultrasound generation member is equal to or greater than one half of the minimum receiving wavelength λ of the ultrasound. When a member having the Young's modulus greater than 0.1 GPa (e.g., plastic film) is used as the ultrasound generation member of the member which becomes the plane sound source, a decrease in accuracy of an image caused by the shear wave propagation inside the ultrasound generation member can be reduced by reducing the thickness of the film. Specifically, in a case in which the valid receiving frequency of the ultrasound receiving array 17 includes 2 MHz, if the thickness of the polyethylene film is 50 m, the influence of a decrease in transmittance of the ultrasound due to a phase change and the total reflection in the shear wave propagation by the ultrasound generation member can be reduced to a negligible extent.

The ultrasound generation member 13a of the present embodiment is disposed in the acoustic liquid 40, such as water and gel, at a position spaced from the subject 16. Therefore, the ultrasound generation member 13a is desirably a material at least harder than the acoustic liquid 40, such as water and gel. It is necessary that the ultrasound generation member 13a is a material having a certain degree of rigidity and desirably is a material at least having the Young's modulus of 0.0001 GPa or greater which is the Young's modulus of general gel as expressed by the following Expression (9).

E>0.0001 GPa (9)

The effect produced by arranging the ultrasound generation member 13a at a position spaced from the subject 16 is described. Upon contact with the subject 16, the ultrasound generation member 13a can be deformed. Since the wave surface of the ultrasound to be generated varies depending on the shape of the ultrasound generation member 13a, deformation of the ultrasound generation member 13a causes a change in the wave surface of the ultrasound. At the time of image reconstruction by the signal processor 19, information about the wave surface of the ultrasound which enters the subject 16 is necessary. Therefore, if the ultrasound generation member 13a is deformed and the wave surface of the ultrasound with which the subject 16 is irradiated is also changed, accuracy of the acquired image decreases unless image reconstruction is conducted in consideration of the deformation of the ultrasound generation member 13a. If, however, the ultrasound generation member 13a is disposed at a position spaced from the subject 16, it is not necessary to consider the deformation of the ultrasound generation member 13a that may otherwise be caused by the subject 16.

By measuring the shape of the ultrasound generation member 13a in a state in which the subject 16 is disposed, the deformation caused by the subject 16 of the ultrasound generation member 13a can be considered. However, the wave surface of the ultrasound generated by the ultrasound generation member 13a immediately after generation is disturbed and, if the ultrasound generation member 13a and the subject 16 are in contact with each other, accuracy of an image decreases in a near field of the subject 16. Therefore, even in a case in which the shape of the ultrasound generation member 13a in a state in which the subject 16 is disposed is known, it is considered that an accurate image can be acquired when the ultrasound generation member 13a is positioned spaced from the subject 16.

Tension Member 13b which Keeps Ultrasound Generation Member Stretched at Position Spaced from Subject 16

The ultrasound generation member 13a is disposed at a position spaced from the subject 16. In the present embodiment, the ultrasound imaging apparatus includes the tension member 13b. The tension member 13b keeps the ultrasound generation member 13a stretched at a position spaced from the subject 16. Usually, the acoustic liquid 40 is located between the subject 16 and the ultrasound receiving array 17 in order to efficiently receive the ultrasound scattered from the subject 16. The acoustic liquid 40 is, for example, water or gel. The ultrasound generation member 13a is also disposed in the acoustic liquid 40. Any tension member 13b may be used as long as it keeps the ultrasound generation member 13a stretched in the acoustic liquid 40 at a position spaced from the subject 16. Specifically, when the ultrasound generation member 13a is a sheet-shaped member, the tension member 13b is a pole, hook, and the like for fixing the ultrasound generation member 13a to a support member 17a of the ultrasound receiving array 17. The tension member 13b is not disposed between an area in which the irradiated area 13c is projected to the subject 16 in the direction parallel to the optical axis c and an area in which the irradiated area 13c is projected to the ultrasound receiving array 17 in the direction parallel to the optical axis c. For example, if the sheet-shaped ultrasound generation member 13a is stretched by a sheet-shaped tension member 13b (here, the irradiated area 13c is in contact with the tension member 13b), the sheet-shaped tension member 13b is also considered to constitute the ultrasound generation member 13a. The tension member 13b may keep the ultrasound generation member 13a stretched in a state immersed in the acoustic liquid 40, or keep a membrane-shaped ultrasound generation member 13a stretched to separate the acoustic liquid on the subject 16 side and the acoustic fluid 40 on the ultrasound receiving array 17 side. The ultrasound generation member 13a may be stretched so as to be inserted in or removed from valid receiving areas of the ultrasound receiving array 17. For example, the sheet-shaped ultrasound generation member 13a may be stretched using two facing wires which are taken up by two facing rollers. The relative positions of the ultrasound generation member 13a and the ultrasound receiving array 17 may be fixed. In this case, by making the ultrasound generation member 13a semitransmissive to the wavelength of the pulsed light source, ultrasound echo measurement and photoacoustic measurement can be conducted in a single observation system using a difference in propagation time from the subject 16.

### Subject 16 and Scatterer 15

Although not constituting a part of the ultrasound imaging apparatus of the present invention, the subject 16 and the scatterer 15 are described below. The ultrasound imaging apparatus of the present invention is used mainly for the diagnosis of malignant tumors, progress observation of chemical treatment, and the like of humans and animals. Regarding the subject 16, the breast, the finger, the hand, the foot, and the like of a living body, in particular, humans and animals are assumed as a target of diagnosis. The ultrasound scatterer 15 inside the subject 16 is those with relatively high acoustic impedance inside the subject 16 or having an acoustic impedance difference from the surroundings. For example, the ultrasound scatterer 15 is calcium carbonate, a fat layer, a mammary gland layer and the like in a tumor, if the human body is a measurement target.

### Ultrasound Receiving Array 17

The ultrasound receiving array 17 which is a receiver receiving the ultrasound generated by the ultrasound generation member 13a upon irradiation of the pulsed light 12 detects the ultrasound and converts the ultrasound into electrical signals which are analog signals. Hereafter, the ultrasound receiving array 17 may be referred to as a probe or a transducer. Any ultrasound receiving array may be used as long as it detects ultrasound signals, such as a transducer using a piezoelectric phenomenon, a transducer using resonance of light, and a transducer using a change of capacitance. In the ultrasound receiving array 17 of the present embodiment, a plurality of receiving elements 17b are typically arranged one-dimensionally or two-dimensionally. The ultrasound receiving array 17 includes a plurality of probes arranged in a manner such that acoustic wave receiving surfaces of the probes face mutually different directions so that their valid receiving areas overlap the isocenter. The isocenter is a specific area in which the valid receiving areas of the ultrasound receiving array 17 constituted by a plurality of probes overlap each other and form a high sensitivity area. If an inner surface of the ultrasound receiving array 17 is hemispherical, the center of curvature of the hemisphere coincides with the isocenter. The shapes in which the inner surface of the ultrasound receiving array 17 has the isocenter are quadric surfaces of revolution including paraboloid of revolution, hyperboloid of revolution, and ellipsoid of revolution.

### Signal Collector 18

The photoacoustic imaging apparatus of the present embodiment desirably has the signal collector 18 which amplifies electrical signals acquired from the ultrasound receiving array 17 and converts the electrical signals from analog signals into digital signals. The signal collector 18 is constituted typically by an amplifier, an A/D converter, a field programmable gate array (FPGA) chip, and the like. If a plurality of reception signals are received from the probe, the signal collector 18 desirably processes a plurality of signals simultaneously. This shortens time until an image is formed. The “reception signal” here is a concept which includes both the analog signal acquired from the ultrasound receiving array 17 and the digital signal converted from the analog signal.

### Signal Processor 19

The signal processor 19 converts the reception signal received by each receiving element 17b into acoustic characteristic value information distribution data of the subject 16 by an ultrasound imaging method. The signal processor 19 is typically a workstation and the like, in which an image reconstruction process and the like are performed by the software programmed in advance. Although the image reconstruction process is, for example, typically a back projection method, any image reconstruction may be used in the present invention.

An example of image reconstruction method (back projection method) is described below. If a value of a voxel at a certain position rᵢ is denoted by U(rᵢ), Expressions (10) and (11) hold.

$$[ Math .7 ]$$

$$\begin{matrix}U ( {r}_{i} ) = {Σ}_{n = 0}^{N - 1} s ( {r}_{i} , {τ}_{i , n} ) \\ [ Math .8 ] & ( 10 ) \\ {τ}_{i , n} = \frac{{r}_{1}}{c} + \frac{{r}_{2}}{c} & ( 11 )\end{matrix}$$

In Expressions (10) and (11), s denotes a reception signal, τ denotes time, r₁ denotes the shortest distance between the ultrasound generation member 13a and the voxel, r₂ denotes the distance between the voxel and the receiving element 17b, and c denotes an average acoustic velocity. The acoustic characteristic information distribution is imaged by reconstructing the reception signal s using these Expressions.

### Display Device 20

The display device 20 is a device which displays image data output from the signal processor 19 and typically is, for example, a liquid crystal display. The display device 20 may be provided separately from the ultrasound imaging apparatus of the present invention.

### Example 1

An example of the ultrasound imaging apparatus using the photoacoustic induced ultrasound imaging to which the present embodiment is applied is described. A configuration of Example 1 is described with reference to the apparatus schematic diagram of FIG. 1. In Example 1, a double wave YAG laser excited Ti:sa laser system is used as the light source 11. The laser system is capable of emitting light of a wavelength between 700 to 900 nm at the ultrasound generation member 13a. The laser light is made to irradiate the ultrasound generation member 13a after being extended to about 3 cm in radius using optical fiber and a diffuser. As the ultrasound generation member 13a, a black-colored rubber (isoprene rubber) sheet (70 mm×70 mm in width and 0.5 mm in thickness) is used. Although the optical absorption coefficient of the black-colored rubber sheet is unknown, transmittance of light at a wavelength of 800 nm of the 0.5-mm-thick black-colored rubber sheet is substantially zero. The black-colored rubber sheet is about 0.005 GPa in Young's modulus and about 200×10⁻⁶/K in linear expansion coefficient. As the ultrasound receiving array 17, 512 receiving elements 17b arranged in a spiral shape on the hemispherical support member 17a are used. As the receiving elements 17b, PZT elements of 5 MHz in center frequency, 70% in bandwidth, and 3 mm in diameter are used. The space between the subject 16 and the ultrasound receiving array 17 is filled with water as the acoustic liquid 40. The rubber sheet is fixed to the hemispherical support member 17a by a hook and, as illustrated in FIG. 1, is disposed in the acoustic liquid 40 in a substantially planar shape at a position between the subject 16 and the ultrasound receiving array 17 with a space from a phantom which is the subject 16.

The signal collector 18 has a function to receive all of the data in 512 channels from the ultrasound receiving array 17 simultaneously, and transfer the data to a PC which is the signal processor 19 after amplifying the analog data and converting the analog data into digital data. The subject 16 is a phantom imitating a living body, and is formed from the 1% Intralipid and the ink hardened with agar. A fishing line 0.3 mm in diameter and a plastic ball 0.3 mm in diameter are embedded in the phantom. Each one of the fishing line (wire) and the plastic ball (sphere) is provided as the ultrasound scatterer 15.

In such a system configuration, the ultrasound generation member 13a is irradiated with 800-nm pulsed light emitted from the irradiation portion 21, the phantom is irradiated with the generated ultrasound (the plane wave), and the backscattered ultrasound 14b from the phantom is received by the ultrasound receiving array 17. The reception signals are converted into digital signals by the signal collector 18 and stored in the PC which is the signal processor 19.

Acoustic characteristic value distribution information data related to an acoustic impedance difference is calculated from the stored reception signals by back projection which is an image reconstruction method expressed by Expressions (10) and (11). The acoustic velocity of water which is the acoustic liquid 40 is used as the average acoustic velocity c.

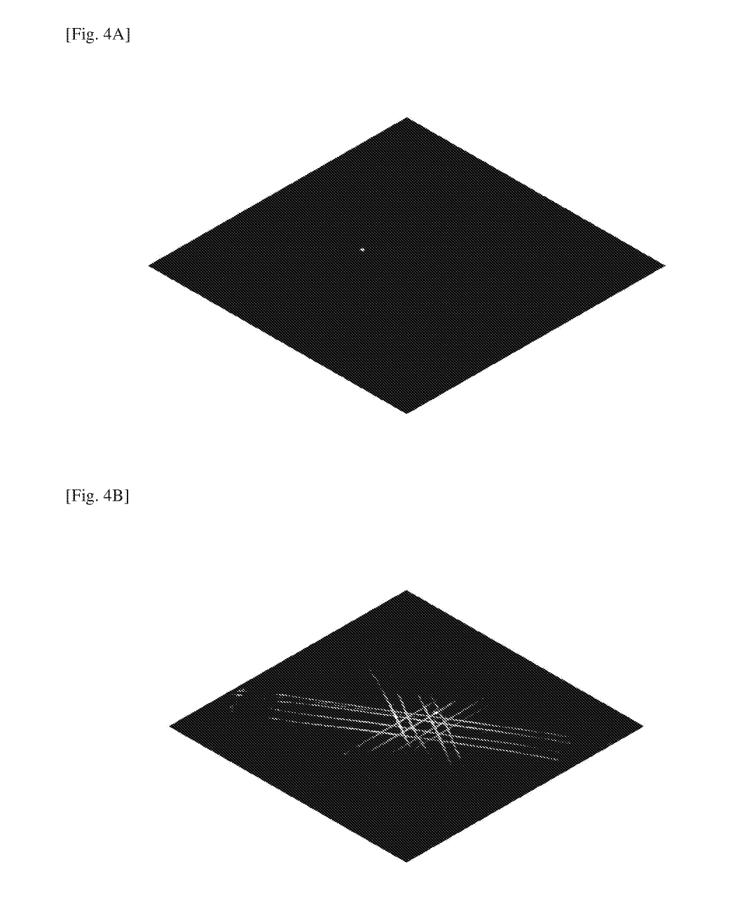

For the comparison, photoacoustic induced ultrasound imaging is conducted in a similar manner using 0.5-mm-thick high-density polyethylene film into which black ink is mixed (Young's modulus: about 1.4 GPa) as an ultrasound generation member. FIG. 4A illustrates a sphere image in a phantom, and FIG. 4B illustrates a wire image in the phantom. FIGS. 4A and 4B are exemplary images acquired when a black-colored rubber sheet is used as the ultrasound generation member 13a. In both the images of FIGS. 4A and 4B, the ultrasound scatterer 15 in the phantom is imaged clearly. When plastic film is used as the ultrasound generation member 13a, the scatterer 15 in the phantom is imaged similarly, but blurring occurs compared with the case in which the black-colored rubber sheet is used, and an image with lower resolution is acquired.

As described above, by disposing the ultrasound generation member which functions as a plane sound source upon light irradiation at a position spaced from the subject, an ultrasound imaging apparatus capable of reducing a decrease in the S/N ratio caused by attenuation of energy of the ultrasound and reducing occurrence of artifact caused by delay of backscattered ultrasound has been provided. In addition, by using a member of which Young's modulus is from 0.0001 to 0.1 GPa as the ultrasound generation member, generation of a shear wave of the ultrasound can be reduced. Therefore, an ultrasound imaging apparatus capable of reducing generation of blurring has been provided without using image reconstruction in consideration of a complicated physical phenomenon.

### Example 2

The basic configuration of the apparatus according to Example 2 is the same as that of the Example 1, and includes a light source 11, an ultrasound generation member 13a, a tension member 13b which keeps the ultrasound generation member 13a stretched at a position spaced from a subject 16, an ultrasound receiving array 17, and a signal processor 19. Example 2 differs from Example 1 in that a high density polyethylene sheet colored in black is used as the ultrasound generation member 13a. The polyethylene sheet is 70 mm×70 mm in width, about 0.05 mm in thickness, and about 1.4 GPa in Young's modulus. Since configurations other than the ultrasound generation member 13a, i.e., the ultrasound receiving array 17, are the same as those of Example 1, description thereof is omitted. As in Example 1, the polyethylene sheet is fixed to a hemispherical support member 17a with a hook and is disposed with a space from a phantom which is the subject 16. If the frequency of the ultrasound which passes through the ultrasound generation member 13a is set to be 6.75 MHz which is the maximum frequency fₘₐₓ of receiving elements 17b and the acoustic velocity of the longitudinal wave of the polyethylene sheet is set to be 2460 m/s, the wavelength of the receivable ultrasound will be about 0.36 mm. Therefore, 0.05 mm which is the thickness of the polyethylene sheet is thinner than one half of the wavelength of the ultrasound propagating through the polyethylene sheet and thus is sufficiently thin.

Acoustic characteristic value distribution information data which is ultrasound image data is generated using the same phantom as that of Example 1 using such an ultrasound imaging apparatus. An image is reconstructed by the same back projection method as in Example 1. Acoustic velocity of water which is the acoustic liquid 40 is used as the acoustic velocity. As a result, substantially the same image as that acquired when the 0.5-mm-thick black-colored rubber sheet is used as the sound generating member described in Example 1 is acquired.

### Example 3

The basic constitution of an apparatus according to Example 3 is the same as those of Examples 1 and 2. Example 3 is the same in configuration as those of Examples 1 and 2 except that a black-colored silicone rubber sheet is used as an ultrasound generation member 13a and that an ultrasound receiving array 17 on which receiving elements 17b are arranged planarly is used. The same configurations are not described repeatedly. The silicone rubber sheet is 70 mm×70 mm in width, about 0.1 mm in thickness, about 0.014 GPa in Young's modulus, about 250×10⁻⁶/K in a linear expansion coefficient, and about 20 mm⁻¹ an optical absorption coefficient. As the ultrasound receiving array 17, a member in which 600 receiving elements 17b are arranged two dimensionally on a plate-shaped tension member is used. As the receiving elements 17b, PZT elements of 3 MHz in center frequency, 70% in bandwidth, and 1 mm in diameter are used. The silicone rubber sheet is disposed at a position spaced from the phantom which is the subject 16 using a gel sheet which is an acoustic liquid 40 as a tension member. In order to image an area wider than the optical irradiated area, the silicone rubber sheet has a curvature so that the silicone rubber sheet is convex to the subject 16, whereby the ultrasound to be generated is diffused slightly. The curvature here is so small that the ultrasound generation member may function as a plane sound source. If the frequency of the ultrasound which passes through the ultrasound generation member 13a is set to be 6.75 MHz which is the maximum frequency fₘₐₓ of receiving elements 17b and the acoustic velocity of the longitudinal wave of the polyethylene sheet is set to be 1485 m/s, the wavelength of the receivable ultrasound will be about 0.22 mm. Therefore, 0.1 mm which is the thickness of the polyethylene sheet is about one half of the wavelength of the ultrasound propagating through the polyethylene sheet.

Acoustic characteristic value distribution information data which is ultrasound image data is acquired using the same phantom as that of Example 1 using such an ultrasound imaging apparatus. The image is reconstructed by the same back projection method as in Example 1. As the average sound velocity c, acoustic velocity of water which is the acoustic liquid 40 is used. As a result, substantially the same image as that of Example 1 is acquired.

Embodiments of the present invention have been described, but the present invention is not limited to the same. Various modifications and changes may be made without departing from the scope of the present invention.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2015-175021, filed Sep. 4, 2015, and No. 2016-148213, filed Jul. 28, 2016 which are hereby incorporated by reference herein in their entirety.

1. An ultrasound receiving apparatus comprising:
an ultrasound generation member configured to generate an ultrasound upon irradiation of light from a light source;
an ultrasound receiving array configured to receive the ultrasound propagating from a subject and output a reception signal; and
a signal processor configured to acquire acoustic characteristic value information about the subject from the reception signal,
wherein the ultrasound generation member is a sheet-shaped light absorbing member which is in acoustic contact with an acoustic liquid disposed between the subject and the ultrasound receiving array so as to acoustically combine the subject and the ultrasound receiving array, and is stretched in the acoustic liquid to be located between the subject and the ultrasound receiving array.

2. The ultrasound receiving apparatus according to claim 1,
wherein the ultrasound receiving array includes a plurality of probes arranged in a manner such that acoustic wave receiving surfaces of the probes face mutually different directions so that their valid receiving areas overlap an isocenter, and the sheet-shaped light absorbing member is disposed so that virtual lines each connecting the isocenter and a center of each of the acoustic wave receiving surfaces of the ultrasound receiving array pass through the sheet-shaped light absorbing member.

3. The ultrasound receiving apparatus according to claim 1,
wherein a diameter of an imaginary circle inscribed in a virtual irradiation surface which is an irradiated area irradiated with light in the ultrasound generation member orthogonally projected onto a virtual plane of which irradiation optical axis is the normal line is 10 or more times of the thickness in the direction parallel to the irradiation optical axis.

4. The ultrasound receiving apparatus according to claim 1, wherein the irradiated area of the ultrasound generation member has the Young's modulus of 0.0001 GPa or greater.

5. The ultrasound receiving apparatus according to claim 1, wherein the irradiated area of the ultrasound generation member has the Young's modulus of 0.1 GPa or less.

6. The ultrasound receiving apparatus according to claim 3, wherein the thickness of the ultrasound generation member is smaller than one half of the minimum receiving wavelength in a valid receiving band of the ultrasound receiving array.

7. The ultrasound receiving apparatus according to claim 1, wherein the ultrasound generation member is rubber.

8. The ultrasound receiving apparatus according to claim 1, wherein the thickness of the ultrasound generation member is greater than an inverse of an optical absorption coefficient of the ultrasound generation member.

9. The ultrasound receiving apparatus according to claim 1, wherein a linear expansion coefficient of the ultrasound generation member is greater than 10×10⁻⁶/K.

10. The ultrasound receiving apparatus according to claim 1, further comprising the light source which irradiates the ultrasound generation member with light.

11. The ultrasound receiving apparatus according to claim 1, wherein the signal processor includes a display unit configured to image acoustic characteristic value information about the subject and display the imaged acoustic characteristic value information about the subject.
